(19) United States
(12) Patent Application Publication
(10) Pub. No.: US 2012/0283190 A1
(43) Pub. Date: Nov. 8, 2012

(54) ENDOTHELIN INHIBITORS FOR THE TREATMENT OF RAPIDLY PROGRESSIVE GLOMERULONEPHRITIS

(75) Inventors: Pierre-Louis Tharaux, Paris (FR); Cecile Fligny, Paris (FR)

(73) Assignee: Institut National De La Santé Et De La Recherche Medicale (INSERM), Paris (FR)

(21) Appl. No.: 13/513,932

(22) PCT Filed: Dec. 8, 2010
(86) PCT No.: PCT/EP2010/069130

§ 371 Date: Jul. 25, 2012

(30) Foreign Application Priority Data
Dec. 9, 2009 (EP) 09306198.4

(51) Int. Cl. A61K 38/06; A61P 13/12; A61K 31/506

(52) U.S. Cl. 514/15.4; 514/269

(57) ABSTRACT

The present invention relates to endothelin inhibitors for use in the treatment of Rapidly Progressive GlomeruloNephritis and to pharmaceutical compositions thereof.

### FIELD OF THE INVENTION

The present invention relates to the use of endothelin inhibitors for the treatment of rapidly progressive glomerulonephritis.

### BACKGROUND OF THE INVENTION

Glomerulonephritis (GN) refers to a heterogeneous group of diseases characterized by inflammatory changes in glomerular capillaries and accompanying signs and symptoms of an acute nephritic syndrome.

Among diseases of this group, Rapidly Progressive GlomeruloNephritis (RPGN), also called crescentic glomerulonephritis or extracapillary glomerulonephritis, consists of the most severe class of glomerulopathies in humans. This disease is a clinical syndrome and a morphological expression of severe glomerular injury. Glomerular injury manifests as a proliferative histological pattern, accumulation of T cells and macrophages, proliferation of intrinsic glomerular cells, accumulation of cells in Bowman's space (“crescents”), and rapid deterioration of renal function. RPGN uusually presents with acute nephritic syndrome (acute renal failure (ARF), fall in urinary output, haematuria, proteinuria, cellular casts in the urine) (Jennette J C et al., 2001; Lionaki S et al., 2007).

The causative agents in most forms of human glomerulonephritis are unknown, but some evidences show that glomerulonephritis follow bacterial or viral infections. Most evidence now suggests that infectious agents, and doubtless other stimuli as well, induce glomerulonephritis by triggering an autoimmune response that results in formation of immune-complex deposits in glomeruli or elicits a cell-mediated immune response to antigens in, or of, the glomerulus (Couser W G, 1998). Other causes include Systemic Lupus Erythematosus, cryoglobulinaemia and Henoch-Schönlein purpura. (Jennette J C et al., 2001; Lionaki S et al., 2007).

Available therapies for RPGN are limited to potent immunosuppressive drugs (high doses of cortico-steroids with cyclophosphamide that can be substituted by azathioprine after remission). In some cases, apheresis therapy is considered in patients with an aggressive form. Approximately one-fourth to one-third of patients experience a recurrence within several years. The need for maintenance immunosuppression therapy raises the problem of adequate immunosuppression versus toxicity. These different immunosuppressive regimens are associated with significant risk of opportunistic infection and sever metabolic side effects (mainly diabetes, dyslipidemia and osteoporosis). On the other hand, some categories of immune complex glomerulonephritis do not necessarily warrant extensive immunosuppressive therapy unless numerous active crescents are present. The prognosis is poor; at least 80% of people develop end-stage kidney failure within six months without treatment. The prognosis is better for people younger than 60 years or if an underlying disorder causing the glomerulonephritis responds to treatment. Surviving patients have an altered quality of life with requirement for side effects-prone non specific immune-suppressants and costly dialysis maintenance.

Thus, there is a need for novel therapies. By defining the conditions and requirements for disease initiation and progression, more specific and directed therapies could be developed for treatment and possibly prevention, which would represent a considerable improvement on current treatment protocols. New treatment permitting the decrease of doses of immunosuppression therapy is of great interest for limiting side effects of such therapy and improving the prognosis.

Some publications (Gomez-Garre D et al., 1996; Ruiz-Ortega M et al. 2001) have already suggested a role of the endothelin pathway in experimental immune glomerulonephritis induced by repeated infusion of ovalbumin in rats. This model of ovalbumin-induced proliferation of mesangial cells has no equivalent in human pathology. Although indirect arguments for activation of ET-1 synthesis in human autoimmune diseases with renal involvement have been occasionally reported (Neuhofer W et al, 2006), no specific study has demonstrated activation of the ET-1 pathway in crescentic glomerulonephritis or RPGN and no beneficial action of modulation of ET-1 actions on the course of this specific syndrome has been demonstrated so far.

In other kidney diseases than RPGN, beneficial action of the blockade of endothelin receptors has been suggested from preclinical models of hypertensive or diabetic condition or nephron reduction, that are important factors in the development of glomerulosclerosis (Neuhofer W et al, 2009), and recently confirmed with short term studies in human subjects with chronic proteinuric glomerulopathies (Dhaun N et al, 2009; Weber M A et al, 2009; Wenzel R R et al, 2009) that do not correspond to the condition of RPGN. Indeed, RPGN is considered as a specific clinical state in which mechanisms differ from other glomerulonephritis. In particular, RPGN is characterized by a proliferation of podocytes (Thorner P S et al, 2008) and parietal epithelial cells (Smeets B et al, 2009) whereas apoptosis of podocytes is generally observed in other glomerulopathies (D'Agati V D, 2008). Thus, the role of endothelin in RPGN was not obvious considering this prior art.

### SUMMARY OF THE INVENTION

The present invention relates to an endothelin inhibitor for use in the treatment of rapidly progressive glomerulonephritis (RPGN).

Particularly, the invention relates to said endothelin inhibitor in combination with an immunosuppressive treatment.

The invention also provides pharmaceutical compositions comprising an endothelin inhibitor of the invention.

### DETAILED DESCRIPTION OF THE INVENTION

The inventors showed, in an experimental model of rapidly progressive glomerulonephritis, that stimulation of endothelin receptors markedly aggravates the pathology and that blockade of endothelin receptors (ETR) prevented nephritic syndrome, infiltration of T cells and macrophages, necrotizing crescentic glomerulonephritis, acute renal failure and death, suggesting that targeting the ETR pathways would be clinically beneficial for treatment of this disease.

### Endothelin Inhibitors

Thus, a first object of the invention relates to an endothelin inhibitor for use in the treatment of rapidly progressive glomerulonephritis (RPGN).

According to the invention, the term Rapidly Progressive Glomerulonephritis (or RPGN) encompasses crescentic glomerulonephritis and extracapillary glomerulonephritis.

The term “treating” or “treatment” refers to reversing, alleviating, inhibiting the progress of, or preventing the disorder or condition to which such term applies, or one or more symptoms of such disorder or condition.

According to the invention, the term “endothelin” has its general meaning in the art and refers to the vasoconstricting peptide endothelin-1 (ET-1) produced primarily in the endothelium and acting on its both receptors ETA and ETB. The endothelin (ET-1) can be from any source, but typically is a mammalian (e.g., human and non-human primate) endothelin, particularly a human endothelin. An exemplary native endothelin-1 amino acid sequence is provided in UniProt database under accession number P05305 and an exemplary native nucleotide sequence encoding for endothelin-1 is provided in GenBank database under accession number NM_{—}001955.

According to the invention, the term “ETA” has its general meaning in the art and refers to the endothelin receptor type A. ETA can be from any source, but typically is a mammalian (e.g., human and non-human primate) ETA, particularly a human ETA. An exemplary native ETA amino acid sequence is provided in UniProt database under accession number P25101 and an exemplary native nucleotide sequence encoding for ETA is provided in GenBank database under accession number NM_{—}001957.

According to the invention, the term “ETB” has its general meaning in the art and refers to the endothelin receptor type B. ETB can be from any source, but typically is a mammalian (e.g., human and non-human primate) ETB, particularly a human ETB. An exemplary native ETB amino acid sequence is provided in UniProt database under accession number P24530 and an exemplary native nucleotide sequence encoding for ETB is provided in GenBank database under accession number NM_{—}000115.

The term “endothelin inhibitor” should be understood broadly and encompasses any substance able to prevent the action of endothelin (ET-1) on its receptors ETA and ETB.

Thus an endothelin inhibitor encompasses an endothelin receptor antagonist of the invention, an inhibitor of endothelin, ETA or ETB gene expression and inhibitor of endothelin formation.

In one embodiment, an endothelin receptor antagonist can be used.

The term “endothelin receptor antagonist” should be understood broadly and encompasses substances acting directly (by binding) on endothelin, ETA or ETB proteins and able to prevent the interaction or binding between endothelin and its receptor(s).

So, said endothelin receptor antagonist particularly encompasses classical endothelin receptor antagonist which are small organic molecules well known in the art. Said molecules may have an antagonist effect on both ETA and ETB receptors ((ETA/ETB antagonist), or only on one of both (selective ETA receptor antagonist or a selective ETB receptor antagonist).

Typically, the endothelin receptor antagonist of the invention can be selected from the group consisting of: darusentan, sitaxsentan, tezosentan, bosentan, macitentan, ambrisentan, atrasentan, avosentan, clazosentan, zibotentan, edonentan, A-182086, A-192621, ABT-627, BMS193884, BQ123, BQ-788, CI1020, FR-139317, S-0139, SB-209670, TA-0201, TAK-44, TBC3711, YM-598, ZD-1611 or ZD4054.

In a particular embodiment of the invention, said endothelin receptor antagonist is an ETA/ETB antagonist.

Particularly, said endothelin receptor antagonist is bosentan (U.S. Pat. No. 5,292,740 and U.S. Pat. No. 5,883,254), described by the following formula:

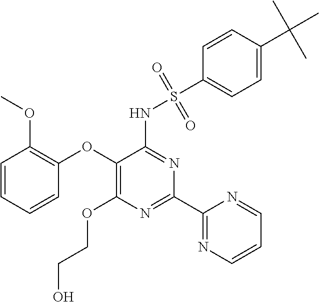

Other compounds can be used, such as macitentan, A-182086, CPUO213, J-104132 or SB209670.

In another particular embodiment of the invention, said endothelin receptor antagonist is a selective ETB receptor antagonist.

A selective ETB receptor antagonist exhibits an affinity (as expressed by dissociation constant Ki) for ETB less than about 10 nM and a selectivity for ETB over ETA (as expressed by the ratio Ki_{ETB}/Ki_{ETA}) is at least about 50. In a particular embodiment, Ki_{ETB} is less than 5 nM, more particularly less than 2 nM and even more particularly less than 1 Nm. In another particular embodiment, the ratio Ki_{ETB}/Ki_{ETA} is at least about 100, more particularly about 500 and even more particularly about 1000.

Particularly, said selective ETB receptor antagonist is BQ-788 (Okada M. et al, 2002), described by the following formula:

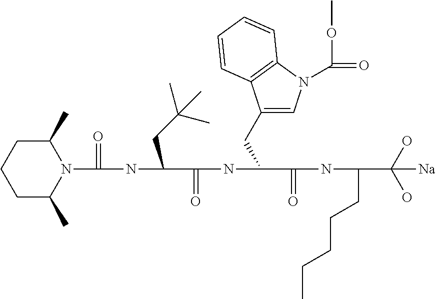

Other compounds can be used, such as A-192621.

An endothelin receptor antagonist may be an antibody or antibody fragment that can partially or completely blocks the interaction between endothelin and its receptors.

In particular, the endothelin receptor antagonist may consist in an antibody directed against endothelin or against ETA and ETB receptors, in such a way that said antibody blocks the binding of endothelin on its receptors.

Antibodies directed against the endothelin or ETA/ETB receptors can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein; the human B-cell hybridoma technique and the EBV-hybridoma technique. Alternatively, techniques described for the production of single chain antibodies (see, e.g., U.S. Pat. No. 4,946,778) can be adapted to produce single chain antibodies of the invention. Endothelin receptor antagonist useful in practicing the present invention also include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity.

Humanized antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In a particular embodiment, said endothelin receptor antagonist is an anti-endothelin antibody.

In another particular embodiment, said endothelin receptor antagonist is an anti-ETB antibody.

Furthermore, the endothelin receptor antagonist may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S D, 1999. Peptide aptamers consists of a conformationally constrained antibody variable region displayed by a platform protein, such as E. coli Thioredoxin A that are selected from combinatorial libraries by two hybrid methods.

In another embodiment, said endothelin inhibitor can be an inhibitor of endothelin gene expression or an inhibitor of ETA or ETB gene expression.

An “inhibitor of expression” refers to a natural or synthetic compound that has a biological effect to inhibit or significantly reduce the expression of a gene. Consequently an “inhibitor of endothelin gene expression” (or “inhibitor of ETA or ETB gene expression” refers to a natural or synthetic compound that has a biological effect to inhibit or significantly reduce the expression of the gene encoding endothelin (or ETA or ETB respectively).

Inhibitors of endothelin (or endothelin receptors) expression for use in the present invention may be based on anti-sense oligonucleotide constructs. Anti-sense oligonucleotides, including anti-sense RNA molecules and anti-sense DNA molecules, would act to directly block the translation of endothelin (or endothelin receptors) mRNA by binding thereto and thus preventing protein translation or increasing mRNA degradation, thus decreasing the level of endothelin, and thus activity, in a cell. For example, antisense oligonucleotides of at least about 15 bases and complementary to unique regions of the mRNA transcript sequence encoding endothelin (or endothelin receptors) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of endothelin (or endothelin receptors) expression for use in the present invention. Endothelin (or endothelin receptors) expression can be reduced by contacting a subject or cell with a small double stranded RNA (dsRNA), or a vector or construct causing the production of a small double stranded RNA, such that endothelin expression is specifically inhibited (i.e. RNA interference or RNAi). Methods for selecting an appropriate dsRNA or dsRNA-encoding vector are well known in the art for genes whose sequence is known (Elbashir S M et al., 2001; Tuschl T et al, 1999; Hannon G J, 2002; Brummelkamp T R et al., 2002; McManus M T et al, 2002) U.S. Pat. Nos. 6,573,099 and 6,506,559; and International Patent Publication Nos. WO 01/36646, WO 99/32619, and WO 01/68836). shRNAs (short hairpin RNA) can also function as inhibitors of endothelin (or endothelin receptors) expression for use in the present invention.

Ribozymes can also function as inhibitors of endothelin (or endothelin receptors) expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of endothelin (or endothelin receptors) mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of endothelin (or endothelin receptors) expression can be prepared by known methods. These include techniques for chemical synthesis such as, e.g., by solid phase phosphorothioate chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a mean of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing endothelin (or endothelin receptors). Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in 35.

Preferred viruses for certain applications are the adenoviruses and adeno-associated (AAV) viruses, which are double-stranded DNA viruses that have already been approved for human use in gene therapy. Actually at least 12 different AAV serotypes (AAV1 to 12) are known, each with different tissue tropisms. Recombinant AAV are derived from the dependent parvovirus AAV2. The adeno-associated virus type 1 to 12 can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion and most recombinant adenovirus are extrachromosomal.

Other vectors include plasmid vectors. Plasmid vectors have been extensively described in the art and are well known to those of skill in the art. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript, pSIREN. Other plasmids are well known to those of ordinary skill in the art. Additionally, plasmids may be custom designed using restriction enzymes and ligation reactions to remove and add specific fragments of DNA. Plasmids may be delivered by a variety of parental, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter.

In a particular embodiment, said endothelin inhibitor is an inhibitor of endothelin gene expression. In another particular embodiment, said endothelin inhibitor is an inhibitor of ETB gene expression.

In a further embodiment, endothelin inhibitor of the invention can be a molecule that decreases or prevents endothelin formation.

Such molecules are particularly Endothelin-Converting Enzyme inhibitor (ECE inhibitor), such as FR901533 (also called WS79089B, Tsurumi Y, et al., 1994 and Tsurumi Y, et al., 1995) or CGS 26303 (De Lombaert S et al, 1994).

In another further embodiment, the use of an inhibitor of Endothelin-Converting Enzyme (ECE) gene expression can also be envisaged.

In a particular embodiment, the endothelin inhibitor of the invention may be used in combination with immunosuppressive treatment for the treatment of RPGN.

Said immunosuppression treatment corresponds to classical immunosuppression treatment which generally consists of oral steroids in combination with oral cyclophosphamide or azathioprine.

### Methods for treatment

A further object of the invention relates to methods and compositions for treatment of a subject afflicted with or susceptible to be afflicted with rapidly progressive glomerulonephritis (RPGN).

In one embodiment, the invention relates to a method for treating RPGN comprising administering a subject in need thereof with a therapeutically effective amount of an endothelin inhibitor of the invention.

The term “subject” denotes a mammal, such as a rodent, a feline, a canine, and a primate. Preferably, a subject according to the invention is a human.

Preferably, said inhibitor is administered in a therapeutically effective amount. By a “therapeutically effective amount” is meant a sufficient amount of the endothelin inhibitor to treat and/or to prevent crescentic glomerulonephritis at a reasonable benefit/risk ratio applicable to any medical treatment.

In a particular embodiment, the invention relates to a method for treating RPGN comprising administering a subject in need thereof with a therapeutically effective amount of an endothelin inhibitor of the invention in combination with classical immunosuppressive treatment, currently used for the treatment of crescentic glomerulonephritis.

The endothelin inhibitor may be administered in the form of a pharmaceutical composition, as defined below.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions

A further object of the invention relates to a pharmaceutical composition for treating and/or preventing RPGN, said composition comprising an endothelin inhibitor of the invention.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The endothelin inhibitor(s) may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The endothelin inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active substances in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The endothelin inhibitor of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Effects of bosentan on albuminuria induced by experimental glomerulonephritis.

FIG. 2: Effects of bosentan on crescentic glomerular damage.

FIG. 3: effects of several endothelin antagonists (selective or not) on podocytes migration in vitro (BQ123 is a selective ETA antagonist; BQ788 is a selective ETB antagonist).

FIG. 4: effect of selective ETA or ETB antagonists (BQ 123 and BQ788 respectively) on albuminuria induced by experimental glomerulonephritis

FIG. 5: effect of selective ETA or ETB antagonists (BQ 123 and BQ788 respectively) preventing increased plasma urea concentration as an index of renal failure.

### EXAMPLE

### Material & Methods

Animals.

Male and female C57B1/6 mice weighing 20-25 g, 10 weeks old, purchased from Charles River France, were housed in a room with a 12 h light-dark cycle with water and food ad libido. Animal care and research protocols were in accordance with the principles and guidelines adopted by French Ministry for Agriculture. The animals were sacrificed by injection of pentobarbital (100 mg/kg i.p.)

Induction of Crescentic Glomerulonephritis and Pharmacological Inhibition.

The accelerated anti-GBM RPGN model was used, as previously described in Topham P S et al, 1999 and Lloyd C M et al, 1997. Briefly, C57B1/6J male mice were given subcutaneous injections of 200 μg normal sheep IgG (100 μl into each flank of normal sheep IgG (Sigma) diluted to 1 mg/ml in a solution of 50% Freund's complete adjuvent and 50% saline). Six days later (day 1), GN was induced by intravenous injection of 50 μL sheep anti-glomerular basement membrane (GBM) serum diluted to 70 mg/ml in saline. Serum injections were repeated twice (on days 2 and 3), with the serum volume increased to 250 μL. From days 0 to 14, mice were given bosentan, an orally active endothelin antagonist (designated chemically as 4-tert-butyl-N-[6-(2-hydroxy-ethoxy)-5-(2-methoxy-phenoxy)-[2,2′]-bipyrimidin-4-yl]-benzenesulfonamide monohydrate). We manually grinded 62.5 mg film-coated tablets of TRACLEER® (bosentan) for oral administration and mixed them with powdered food supplied to the animals at an active dose of 50 mg/kg/d for 14 days as previously described³. Each TRACLEER® 62.5 mg tablet contains 64.541 mg of bosentan, equivalent to 62.5 mg of anhydrous bosentan. The chosen dose is about the maximum recommended human dose [MRHD], on a mg/m² basis. The treated group (n=7) was compared to vehicle-treated animals (n=6). On day 14, mice were sacrificed, and blood was obtained by aortic catheterization; the kidneys were dissected out from each mouse and processed for immunohistochemistry, protein and mRNA analysis, and the remaining half-kidney fixed in 10% neutral-buffered formalin for histology.

In a second set of experiments, 12 weeks old C57B1/6J females were implanted with subcutaneous osmotic minipumps (model 2004, Alzet, Calif.) and randomly assigned to three experimental conditions with continuous infusion of vehicle (PBS, n=11), the ETA selective antagonist BQ 123 sodium salt (A.G. Scientific, Inc.) (n=6) or the ETB selective antagonist BQ 788 (Calbiochem) (n=5) at the dose of 100 nmol·kg⁻¹·day⁻¹ each. The same experimental model of RPGN was applied and the animals were sacrificed on day 17.

Histopathology and Physiological Assessments.

Normalized albumin urinary excretion was evaluated from urine samples collected for 18 h in metabolic cages (Techniplast). Urinary creatinine and albumin were quantified spectrophotometrically using colorimetric methods (Olympus).

For light microscopy, half mouse kidneys were fixed and embedded in paraffin, and 3 μm-thick sections were cut. Histopathological changes were evaluated using Masson's trichrome coloration by an examiner who was blind to the experimental conditions. The proportion of crescentic glomeruli was determined by examination of at least 50 glomeruli per section.

In Vitro Assays in Cultured Podocytes.

Selected experiments (migration studies) were performed in a murine podocyte cell line (Schiwek D et al, 2004) derived from isolated from kidneys of Immorto-Mouse® mice (Charles River, St. Louis, Mo., USA), carrying a temperature-sensitive mutant of the immortalizing SV40 large T antigen under control of the interferon-gamma (INF-γ)-inducible H-2 Kb promoter. Migration assays were performed in μ-Dish 35 mm high with Culture-Insert (Ibidi). Ibidi's wounding inserts were used for cell migration or cell invasion studies. They are more effective than the traditional scrape method because they leave a well-defined cell free gap. The inserts can be inserted into any bio-compatible surface and used to culture one or multiple cell lines. These inserts have two wells that create 2 distinct cell patches with a defined gap of 500 μm. The coverage of this gap after 20 hours of culture was assessed in the presence of the selective ETA antagonist BQ123 or the ETB antagonist BQ 788 (Calbiochem) or vehicle (RPMI with DMSO).

Results:

Effects of Endothelin Antagonism on Experimental Model of RPGN.

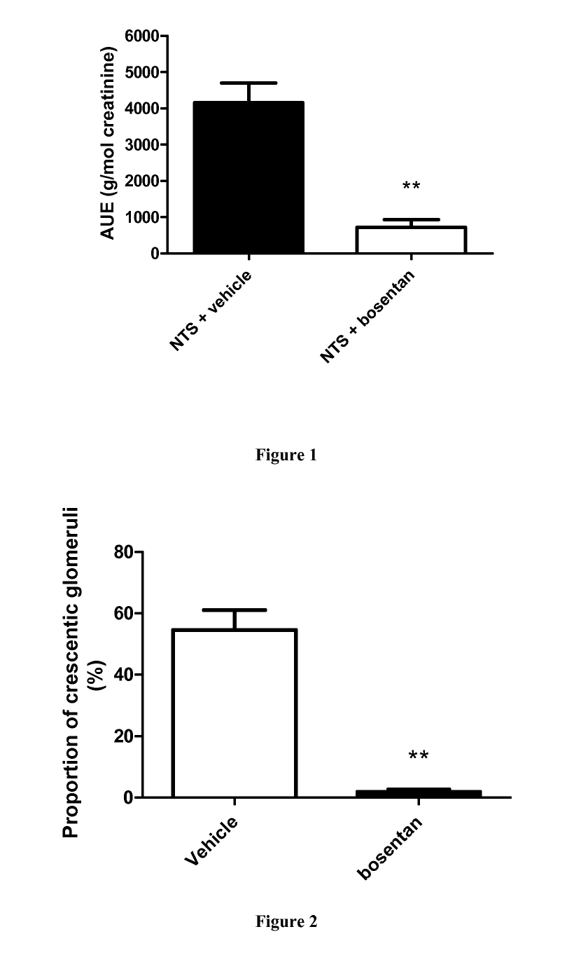

Experimental RPGN provoked marked albuminuria whereas albuminuria above 5 μg/mol creatinine was undetectable in normal mice at baseline. Albuminuria normalized to creatininuria was fivefold (5.8) lower in bosentan-treated animals than in their vehicle-treated counterparts (p=0.0053) on day 5 after infusion of the anti-GBM serum (FIG. 1; **: p<0.01).

Experimental RPGN also provoked crescentic damage in more than half of the glomeruli. Bosentan-treated animals displayed 14 fold less crescentic glomeruli than in their vehicle-treated counterparts on day 14 after infusion of the anti-GBM serum (FIG. 2; p=0.0057; **: p<0.01).

Whereas normal mouse kidneys are devoid of any fibrinogen deposition, mice with anti-GBM serum-induced RPGN displayed marked fibrinogen deposition in glomeruli with disappearance of podocin positive podocytes. These characteristic features of severe RPGN were blunted in mice treated with an endothelin receptor antagonist, bosentan.

Effects of Specific ETA or ETB Antagonism on Experimental Model of RPGN.

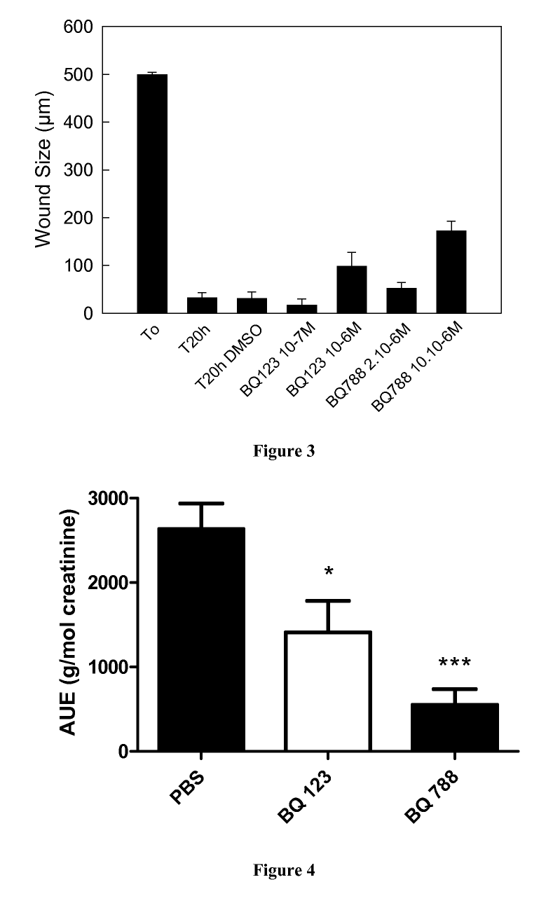

Autocrine endothelin induces a migratory phenotype in podocytes in vitro. The migration of cultured podocytes was significantly blunted by ETR antagonists with a prominent action of the ETB antagonist BQ 788 (FIG. 3).

Albuminuria normalized to creatininuria was blunted in ERA-treated animals than in their vehicle-treated counterparts on day 7 after infusion of the anti-GBM serum (*: p<0.05; ***: p<0.001 versus PBS-treated animals). In particular, a prominent action of the ETB antagonist BQ 78 was measured, although ETA antagonist BQ123 has also a significant effect (FIG. 4).

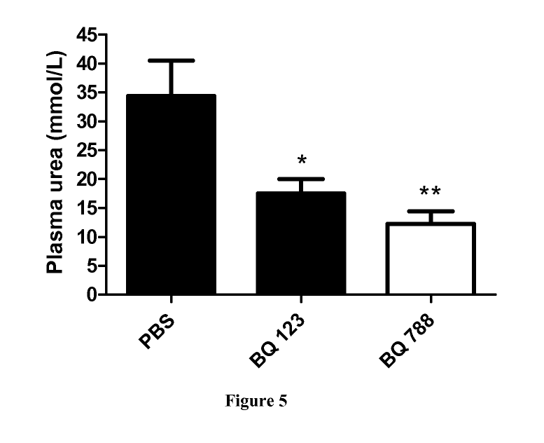

Vehicle only-treated animals developed severe renal failure as assessed by plasma urea concentration (34.3±6.1 mmol/L). The animals treated with an endothelin receptor antagonist developed fewer or no renal failure (17.5±2.5 and 12.3±2.1 mmol/L for BQ123- and BQ788-treated animals, respectively). (*: p<0.05; **: p<0.01 versus PBS-treated animals) (FIG. 5).

### REFERENCES

Throughout this application, various references describe the state of the art to which this invention pertains. The disclosures of these references are hereby incorporated by reference into the present disclosure.
Couser W G. Pathogenesis of glomerular damage in glomerulonephritis. Nephrol Dial Transplant. 1998; 13 Suppl 1:10-5.
D'Agati V D. The spectrum of focal segmental glomerulosclerosis: new insights. Curr Opin Nephrol Hypertens. 2008 May; 17(3):271-81.
De Lombaert S, Ghai R D, Jeng A Y, Trapani A J, Webb R L. Pharmacological profile of a non-peptidic dual inhibitor of neutral endopeptidase 24.11 and endothelin-converting enzyme. Biochem Biophys Res Commun. 1994 Oct. 14; 204(1):407-12.
Dhaun N, Macintyre I M, Melville V, Lilitkarntakul P, Johnston N R, Goddard J, Webb D J. Blood pressure-independent reduction in proteinuria and arterial stiffness after acute endothelin-a receptor antagonism in chronic kidney disease. Hypertension. 2009 July; 54(1):113-9.
Gómez-Garre D, Largo R, Liu X H, Gutierrez S, Lopez-Armada M J, Palacios I, Egido J. An orally active ETA/ETB receptor antagonist ameliorates proteinuria and glomerular lesions in rats with proliferative nephritis. Kidney Int. 1996 September; 50(3):962-72.

Jennette J C, Thomas D B. Crescentic glomerulonephritis. Nephrol Dial Transplant. 2001; 16 Suppl 6:80-82.

Lionaki S, Jennette J C, Falk R J. Anti-neutrophil cytoplasmic (ANCA) and anti-glomerular basement membrane (GBM) autoantibodies in necrotizing and crescentic glomerulonephritis. Semin Immunopathol. 2007; 29(4):459-474.
Lloyd C M, Minto A W, Dorf M E, Proudfoot A, Wells T N, Salant D J, Gutierrez-Ramos J C. RANTES and monocyte chemoattractant protein-1 (MCP-1) play an important role in the inflammatory phase of crescentic nephritis, but only MCP-1 is involved in crescent formation and interstitial fibrosis. J Exp Med. 1997; 185(7):1371-1380.
Neuhofer W, Pittrow D. Endothelin in human autoimmune diseases with renal involvement. Rheumatology (Oxford). 2006 October; 45 Suppl 3:iii39-41.
Neuhofer W, Pittrow D. Endothelin receptor selectivity in chronic kidney disease: rationale and review of recent evidence. Eur J Clin Invest. 2009 June; 39 Suppl 2:50-67.
Okada M, Nishikibe M. BQ-788, a selective endothelin ET(B) receptor antagonist. Cardiovasc Drug Rev. 2002 Winter;20(1):53-66.
Ruiz-Ortega M, Gómez-Garre D, Liu X H, Blanco J, Largo R, Egido J. Quinapril decreases renal endothelin-1 expression and synthesis in a normotensive model of immune-complex nephritis. J Am Soc Nephrol. 1997 May; 8(5):756-68.
Schiwek D, Endlich N, Holzman L, Holthofer H, Kriz W, Endlich K. Stable expression of nephrin and localization to cell-cell contacts in novel murine podocyte cell lines. Kidney Int. 2004; 66(1):91-101.
Smeets B, Angelotti M L, Rizzo P, Dijkman H, Lazzeri E, Mooren F, Ballerini L, Parente E, Sagrinati C, Mazzinghi B, Ronconi E, Becherucci F, Benigni A, Steenbergen E, Lasagni L, Remuzzi G, Wetzels J, Romagnani P. Renal Progenitor Cells Contribute to Hyperplastic Lesions of Podocytopathies and Crescentic Glomerulonephritis. J Am Soc Nephrol. 2009 Nov. 19.
Thorner P S, Ho M, Eremina V, Sado Y, Quaggin S. Podocytes contribute to the formation of glomerular crescents. J Am Soc Nephrol. 2008 March; 19(3):495-502.
Topham P S, Csizmadia V, Soler D, Hines D, Gerard C J, Salant D J, Hancock W W. Lack of chemokine receptor CCR1 enhances Th1 responses and glomerular injury during nephrotoxic nephritis. J Clin Invest. 1999; 104(11):1549-1557.
Tsurumi Y, Ohhata N, Iwamoto T, Shigematsu N, Sakamoto K, Nishikawa M, Kiyoto S, Okuhara M. WS79089A, B and C, new endothelin converting enzyme inhibitors isolated from Streptosporangium roseum. No. 79089. Taxonomy, fermentation, isolation, physico-chemical properties and biological activities. J Antibiot (Tokyo). 1994 June; 47(6):619-30.
Tsurumi Y, Fujie K, Nishikawa M, Kiyoto S, Okuhara M. Biological and pharmacological properties of highly selective new endothelin converting enzyme inhibitor WS79089β isolated from Streptosporangium roseum No. 79089. J Antibiot (Tokyo). 1995 February; 48(2):169-74.
Weber M A, Black H, Bakris G, Krum H, Linas S, Weiss R, Linseman J V, Wiens B L, Warren M S, Lindholm L H. A selective endothelin-receptor antagonist to reduce blood pressure in patients with treatment-resistant hypertension: a randomised, double-blind, placebo-controlled trial. Lancet. 2009 Oct. 24; 374(9699):1423-31.
Wenzel R R, Littke T, Kuranoff S, Jürgens C, Bruck H, Ritz E, Philipp T, Mitchell A; SPP301 (Avosentan) Endothelin Antagonist Evaluation in Diabetic Nephropathy Study Investigators. Avosentan reduces albumin excretion in diabetics with macroalbuminuria. J Am Soc Nephrol. 2009 March; 20(3):655-64.

1-10. (canceled)

11. A method of treating rapidly progressive glomerulonephritis in a patient in need thereof, comprising the step of
administering to said patient a therapeutically effective amount of an endothelin inhibitor.

12. The method of claim 11, wherein said endothelin inhibitor is an endothelin receptor antagonist.

13. The method of claim 12, wherein said endothelin receptor antagonist is an endothelin receptor type A/endothelin receptor type B (ETA/ETB) antagonist.

14. The method of claim 13, wherein said ETA/ETB antagonist is bosentan.

15. The method of claim 12, wherein said endothelin receptor antagonist is a selective ETB antagonist.

16. The method of claim 15, wherein said selective ETB antagonist is BQ-788.

17. The method of claim 11, wherein said endothelin inhibitor is an inhibitor of endothelin expression.

18. The method of claim 12, wherein said antagonist is an inhibitor of ETA or ETB expression.

19. The method of claim 11, wherein said endothelin inhibitor is an Endothelin Converting Enzyme inhibitor.

20. The method of claim 11, further comprising the step of administering to said patient an immunosuppressive treatment.
